(19) United States
(12) Patent Application Publication
(10) Pub. No.: US 2011/0144658 A1
(43) Pub. Date: Jun. 16, 2011

(54) CATHETER SIMULATION AND ASSISTANCE SYSTEM

(75) Inventors: Tal Wenderow, West Newton, MA (US); Thomas Bromander, Andover, MA (US)

(73) Assignee: Corindus Inc.

(21) Appl. No.: 13/034,618

(22) Filed: Feb. 24, 2011

Related U.S. Application Data
(63) Continuation of application No. PCT/US09/55318, filed on Aug. 28, 2009.
(60) Provisional application No. 61/093,229, filed on Aug. 29, 2008.

(51) Int. Cl. A61B 19/00

(52) U.S. Cl. 606/130

(57) ABSTRACT

A workstation configured for operating and simulating a robotic catheter system and interventional procedure is provided. The workstation includes a user interface configured to receive a user input and a control system operatively coupled to the user interface for remotely and independently controlling at least two percutaneous intervention devices. The workstation includes a data storage subsystem to store a vascular image data and a simulation subsystem. The control system controls the at least two percutaneous intervention devices based upon the user input received by the user interface to allow the user to perform a real catheterization procedure. The simulation subsystem generates a display as a function of the vascular image data and causes the display of a simulated percutaneous intervention device within the vascular image data as a function of the user input allowing the user to perform a simulated catheterization procedure.

### CROSS-REFERENCE TO RELATED PATENT APPLICATIONS

The present application is a continuation of prior International Application No. PCT/US09/055318, filed Aug. 28, 2009, which claims the benefit of U.S. Provisional Patent Application No. 61/093,229, filed on Aug. 29, 2008, both of which are incorporated herein by reference in their entireties.

### BACKGROUND OF THE INVENTION

The present invention relates generally to the field of catheter systems for performing diagnostic and/or intervention procedures. The present invention relates specifically to catheter systems including simulation and/or assistance functionality.

Vascular disease, and in particular cardiovascular disease, may be treated in a variety of ways. Surgery, such as cardiac bypass surgery, is one method for treating cardiovascular disease. However, under certain circumstances, vascular disease may be treated with a catheter based intervention procedure, such as angioplasty. Catheter based intervention procedures are generally considered less invasive than surgery. If a patient shows symptoms indicative of cardiovascular disease, an image of the patient's heart may be taken to aid in the diagnosis of the patient's disease and to determine an appropriate course of treatment. For certain disease types, such as atherosclerosis, the image of the patient's heart may show a lesion that is blocking one or more coronary arteries. Following the diagnostic procedure, the patient may undergo a catheter based intervention procedure. During one type of intervention procedure, a catheter is inserted into the patient's femoral artery and moved through the patient's arterial system until the catheter reaches the site of the lesion. In some procedures, the catheter is equipped with a balloon or a stent that when deployed at the site of a lesion allows for increased blood flow through the portion of the coronary artery that is affected by the lesion. In addition to cardiovascular disease, other disease may be treated with catheterization procedures.

### SUMMARY OF THE INVENTION

One embodiment of the invention relates to a workstation configured for operating and simulating a robotic catheter system and interventional procedure. The workstation includes a user interface configured to receive a user input and a control system operatively coupled to the user interface for remotely and independently controlling at least two percutaneous intervention devices. The workstation includes a data storage subsystem to store a vascular image data and a simulation subsystem. The control system controls the at least two percutaneous intervention devices based upon the user input received by the user interface to allow the user to perform a real catheterization procedure. The simulation subsystem generates a display as a function of the vascular image data and causes the display of a simulated percutaneous intervention device within the vascular image data as a function of the user input allowing the user to perform a simulated catheterization procedure.

Another embodiment of the invention relates to workstation configured for operating and simulating a robotic catheter system and configured for simulating an imaging system movable between a plurality of angular positions relative to a patient to capture a plurality of angular views of a portion of the patient. The workstation includes a user interface configured to receive a user input and a control system operatively coupled to the user interface for remotely and independently controlling at least two percutaneous intervention devices. The workstation also includes a data storage subsystem to store three dimensional patient specific vascular image data and a simulation subsystem. The simulation subsystem displays a plurality of two dimensional images from the three dimensional vascular image data representative of different angular views of a portion of the patient that may be displayed during a real catheterization procedure. The control system controls the at least two percutaneous intervention devices based upon the user input received by the user interface to allow the user to perform a real catheterization procedure.

Another embodiment of the invention relates to a workstation configured for operating and simulating a robotic catheter system and interventional procedure. The workstation includes a user interface configured to receive a user input and a control system operatively coupled to the user interface for remotely controlling at least two percutaneous intervention devices. The workstation also includes a data storage subsystem to store a vascular image data and a simulation subsystem. The control system controls the at least two percutaneous intervention devices based upon the user input received by the user interface to allow the user to perform a real catheterization procedure. The simulation subsystem generates a display as a function of the vascular image data and causes the display of a simulated intervention device as a function of the user input. The display generated by the simulation subsystem may be displayed during a real catheterization procedure.

### BRIEF DESCRIPTION OF THE DRAWINGS

This application will become more fully understood from the following detailed description, taken in conjunction with the accompanying figures, wherein like reference numerals refer to like elements in which:

FIG. 1 is a perspective view of the robotic catheter system according to an exemplary embodiment;

FIG. 2 is block diagram of a robotic catheter system according to an exemplary embodiment;

FIG. 3 is a block diagram of a control and simulation system according to an exemplary embodiment;

FIG. 4 is an image of a human heart shown during a simulated catheter based medical procedure according to an exemplary embodiment;

FIG. 5 is an image of coronary arteries shown during a simulated catheter based medical procedure according to an exemplary embodiment;

FIG. 6 is a diagram of a catheter based procedure including a simulation step according to an exemplary embodiment;

FIG. 7 is an image of a human heart shown during a real catheter based medical procedure according to an exemplary embodiment;

FIG. 8 is an image of coronary arteries shown during a real catheter based intervention procedure according to an exemplary embodiment; and

FIG. 9A and FIG. 9B show a C-arm imaging system positioned at two angular positions relative to a patient.

### DETAILED DESCRIPTION OF THE PREFERRED EMBODIMENTS

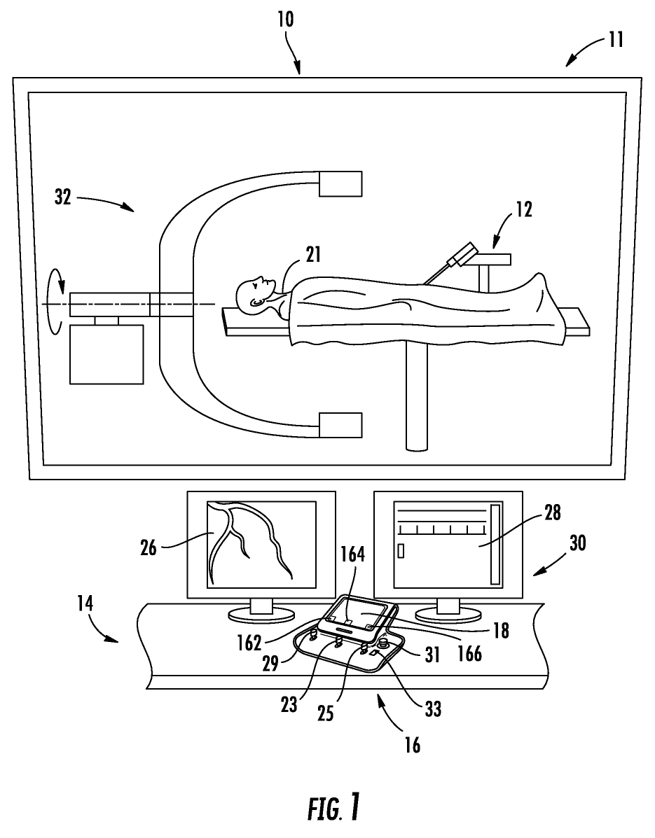

Referring to FIG. 1, a catheter procedure system 10 is shown. Catheter procedure system 10 may be used to perform catheter based medical procedures (e.g., percutaneous intervention procedures). Percutaneous intervention procedures may include diagnostic catheterization procedures during which one or more catheters are used to aid in the diagnosis of a patient's disease. For example, during one embodiment of a catheter based diagnostic procedure, a contrast media is injected into one or more coronary arteries through a catheter and an image of the patient's heart is taken. Percutaneous intervention procedures may also include catheter based therapeutic procedures (e.g., angioplasty, stent placement, treatment of peripheral vascular disease, etc.) during which a catheter is used to treat a disease. It should be noted, however, that one skilled in the art would recognize that, certain specific percutaneous intervention devices or components (e.g., type of guide wire, type of catheter, etc.) will be selected based on the type of procedure that is to be preformed. Catheter procedure system 10 is capable of performing any number of catheter based medical procedures with minor adjustments to accommodate the specific percutaneous intervention devices to be used in the procedure. In particular, while the embodiments of catheter procedure system 10 described herein are explained primarily in relation to the diagnosis and/or treatment of coronary disease, catheter procedure system 10 may be used to diagnose and/or treat any type of disease or condition amenable to diagnosis and/or treatment via a catheter based procedure.

Catheter procedure system 10 includes lab unit 11 and workstation 14. Catheter procedure system 10 includes a robotic catheter system, shown as bedside system 12, located within lab unit 11 adjacent patient 21. Generally, bedside system 12 may be equipped with the appropriate percutaneous intervention devices or components (e.g., guide wires, guide catheters, working catheters, catheter balloons, stents, contrast media, medicine, diagnostic catheters, etc.) to allow the user to perform a catheter based medical procedure. A robotic catheter system, such as bedside system 12, may be any system configured to allow a user to perform a catheter-based medical procedure via a robotic system by operating various controls such as the controls located at workstation 14. Bedside system 12 may include any number and/or combination of components to provide bedside system 12 with the functionality described herein. Various embodiments of bedside system 12 are described in detail in U.S. Provisional Application No. 61/050,933, filed May 6, 2008, which is incorporated herein by reference in its entirety. Other embodiments of bedside system 12 are described in detail in International Application No. PCT/US2009/042720, filed May 4, 2009, which is incorporated herein by reference in its entirety.

In one embodiment, bedside system 12 may be equipped to perform a catheter based diagnostic procedure. In this embodiment, bedside system 12 may be equipped with a variety of catheters for the delivery of contrast media to the coronary arteries. In one embodiment, bedside system 12 may be equipped with a first catheter shaped to deliver contrast media to the coronary arteries on the left side of the heart, a second catheter shaped to deliver contrast media to the coronary arteries on the right side of the heart, and a third catheter shaped to deliver contrast media into the chambers of the heart.

In another embodiment, bedside system 12 may be equipped to perform a catheter based therapeutic procedure. In this embodiment, bedside system 12 may be equipped with a guide catheter, a guide wire, and a working catheter (e.g., a balloon catheter, a stent delivery catheter, etc.). In another embodiment, bedside system 12 may be equipped with an intravascular ultrasound (IVUS) catheter. In another embodiment, any of the percutaneous intervention devices of bedside system 12 may be equipped with positional sensors that indicate the position of the component within the body.

Bedside system 12 is in communication with workstation 14, allowing signals generated by the user inputs of workstation 14 to be transmitted to bedside system 12 to control the various functions of beside system 12. Bedside system 12 also may provide feedback signals (e.g., operating conditions, warning signals, error codes, etc.) to workstation 14. Bedside system 12 may be connected to workstation 14 via a communication link 38 that may be a wireless connection, cable connectors, or any other means capable of allowing communication to occur between workstation 14 and beside system 12.

Workstation 14 includes a user interface 30. User interface 30 includes controls 16. Controls 16 allow the user to control bedside system 12 to perform a catheter based medical procedure. For example, controls 16 may be configured to cause bedside system 12 to perform various tasks using the various percutaneous intervention devices with which bedside system 12 may be equipped (e.g., to advance, retract, or rotate a guide wire, advance, retract, or rotate a working catheter, advance, retract, or rotate a guide catheter, inflate or deflate a balloon located on a catheter, position and/or deploy a stent, inject contrast media into a catheter, inject medicine into a catheter, or to perform any other function that may be performed as part of a catheter based medical procedure).

In one embodiment, controls 16 include a touch screen 18, a dedicated guide catheter control 29, a dedicated guide wire control 23, and a dedicated working catheter control 25. In this embodiment, guide wire control 23 is a joystick configured to advance, retract, or rotate a guide wire, working catheter control 25 is a joystick configured to advance, refract, or rotate a working catheter, and guide catheter control 29 is a joystick configured to advance, retract, or rotate a guide catheter. Controls 16 may also include a balloon or stent control that is configured to inflate or deflate a balloon and/or a stent. Each of the controls may include one or more buttons, joysticks, touch screens, etc. that may be desirable to control the particular component to which the control is dedicated.

Controls 16 may include an emergency stop button 31 and a multiplier button 33. When emergency stop button 31 is pushed a relay is triggered to cut the power supply to bedside system 12. Multiplier button 33 acts to increase or decrease the speed at which the associated component is moved in response to a manipulation of guide catheter control 29, guide wire control 23, and working catheter control 25. For example, if operation of guide wire control 23 advances the guide wire at a rate of 1 mm/sec, pushing multiplier button 33 may cause operation of guide wire control 23 to advance the guide wire at a rate of 2 mm/sec. Multiplier button 33 may be a toggle allowing the multiplier effect to be toggled on and off. In another embodiment, multiplier button 33 must be held down by the user to increase the speed of a component during operation of controls 16.

User interface 30 may include a first monitor 26 and a second monitor 28. First monitor 26 and second monitor 28 may be configured to display information or patient specific data to the user located at workstation 14. For example, first monitor 26 and second monitor 28 may be configured to display image data (e.g., x-ray images, MRI images, CT images, ultrasound images, etc.), hemodynamic data (e.g., blood pressure, heart rate, etc.), patient record information (e.g., medical history, age, weight, etc.). In addition, first monitor 26 and second monitor 28 may be configured to display procedure specific information (e.g., duration of procedure, catheter or guide wire position, volume of medicine or contrast agent delivered, etc.). Further, monitor 26 and monitor 28 may be configured to display information to provide the functionalities associated with the various modules of controller 40 discussed below. In another embodiment, user interface 30 includes a single screen of sufficient size to display one or more of the display components and/or touch screen components discussed herein.

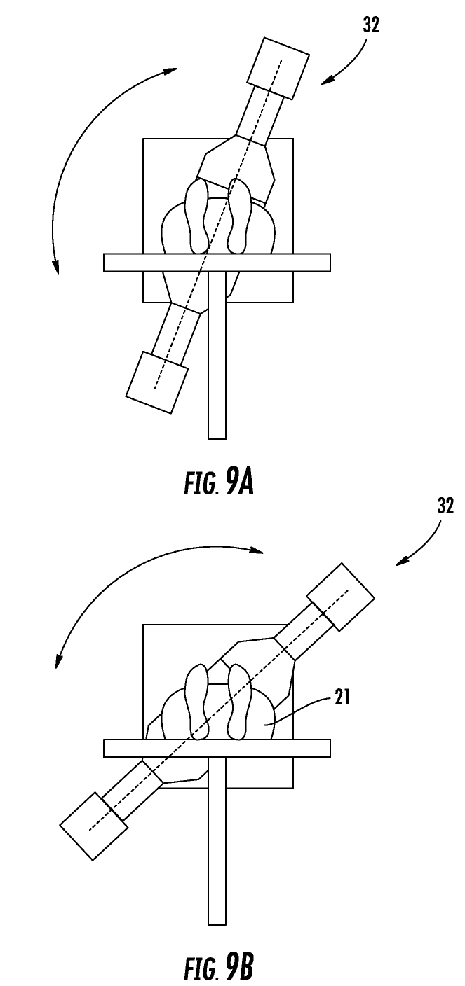

Catheter procedure system 10 also includes an imaging system 32 located within lab unit 11. Imaging system 32 may be any medical imaging system that may be used in conjunction with a catheter based medical procedure (e.g., non-digital x-ray, digital x-ray, CT, MRI, ultrasound, etc.). In an exemplary embodiment, imaging system 32 is a digital x-ray imaging device that is in communication with workstation 14. As shown in FIG. 1, imaging system 32 may include a C-arm that allows imaging system 32 to partially or completely rotate around patient 21 in order to obtain images at different angular positions relative to patient 21 (e.g., sagital views, caudal views, cranio-caudal views, etc.). FIG. 9a and FIG. 9b shows imaging system 32 at two different angular positions relative to patient 21.

Imaging system 32 is configured to take x-ray images of the appropriate area of patient 21 during a particular procedure. For example, imaging system 32 may be configured to take one or more x-ray images of the heart to diagnose a heart condition. Imaging system 32 may also be configured to take one or more x-ray images during a catheter based medical procedure (e.g., real-time images) to assist the user of workstation 14 to properly position a guide wire, catheter, stent, etc. during the procedure. The image or images may be displayed on first monitor 26 and/or second monitor 28. In addition, the user of workstation 14 may be able to control the angular position of imaging system 32 relative to the patient to obtain and display various views of the patient's heart on first monitor 26 and/or second monitor 28. Displaying different views at different portions of the procedure may aid the user of workstation 14 properly move and position the percutaneous intervention devices within the 3D geometry of the patient's heart. In an exemplary embodiment, imaging system 32 may be any 3D imaging modality of the past, present, or future, such as an x-ray based computed tomography (CT) imaging device, a magnetic resonance imaging device, a 3D ultrasound imaging device, etc. In this embodiment, the image of the patient's heart that is displayed during a procedure may be a 3D image. In addition, controls 16 may also be configured to allow the user positioned at workstation 14 to control various functions of imaging system 32 (e.g., image capture, magnification, collimation, c-arm positioning, etc.).

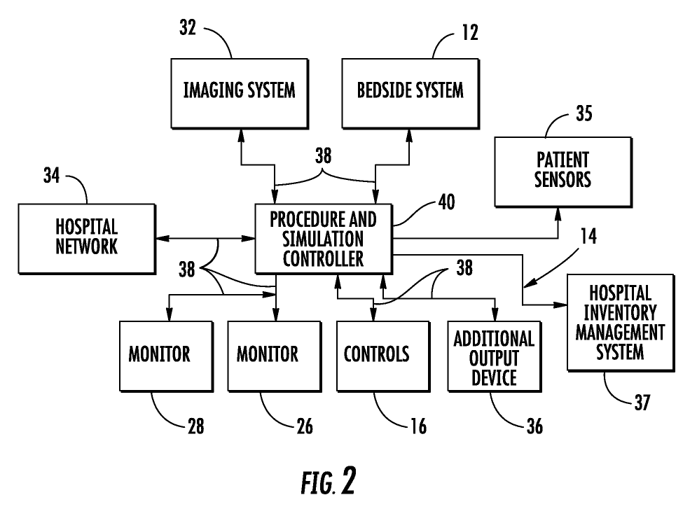

Referring to FIG. 2, a block diagram of catheter procedure system 10 is shown according to an exemplary embodiment. Catheter procedure system 10 may include a control system, shown as procedure and simulation controller 40. As shown in FIG. 2, controller 40 may be part of workstation 14. Controller 40 is in communication with one or more bedside systems 12, controls 16, monitors 26 and 28, imaging system 32, and patient sensors 35 (e.g., electrocardiogram (“ECG”) devices, electroencephalogram (“EEG”) devices, blood pressure monitors, temperature monitors, heart rate monitors, respiratory monitors, etc.). In addition, controller 40 may be in communication with a hospital data management system or hospital network 34, one or more additional output devices 36 (e.g., printer, disk drive, cd/dvd writer, etc.), and a hospital inventory management system 37. Communication between the various components of catheter procedure system 10 may be accomplished via communication links 38. Communication links 38 may be dedicated wires or wireless connections. Communication links 38 may also represent communication over a network. Catheter procedure system 10 may be connected or configured to include any other systems and/or devices not explicitly shown. For example, catheter procedure system 10 may include IVUS systems, image processing engines, data storage and archive systems, automatic balloon and/or stent inflation systems, contrast media and/or medicine injection systems, medicine tracking and/or logging systems, user logs, encryption systems, systems to restrict access or use of catheter procedure system 10, robotic catheter systems of the past, present, or future, etc.

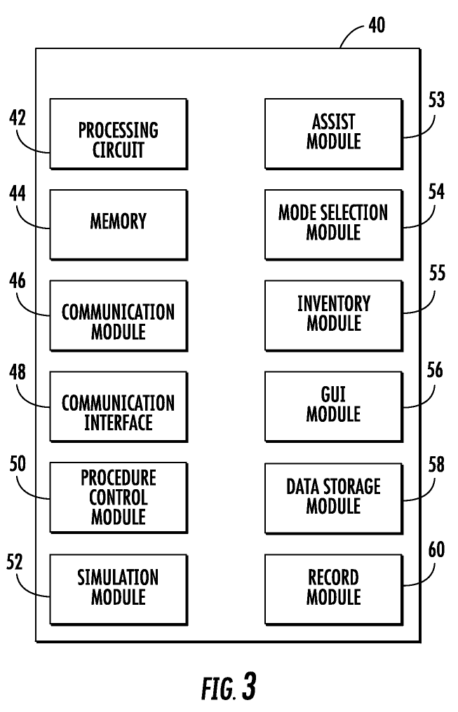

Referring to FIG. 3, a block diagram of controller 40 is shown according to an exemplary embodiment. Controller 40 may generally be an electronic control unit suitable to provide catheter procedure system 10 with the various functionalities described herein. For example, controller 40 may be an embedded system, a dedicated circuit, a general purpose system programmed with the functionality described herein, etc. Controller 40 includes a processing circuit 42, memory 44, communication module or subsystem 46, communication interface 48, procedure control module or subsystem 50, simulation module or subsystem 52, assist module or subsystem 53, mode selection module or subsystem 54, inventory module or subsystem 55, GUI module or subsystem 56, data storage module or subsystem 58, and record module or subsystem 60.

Processing circuit 42 may be a general purpose processor, an application specific processor (ASIC), a circuit containing one or more processing components, a group of distributed processing components, a group of distributed computers configured for processing, etc. configured provide the functionality of module or subsystem components 46, 50-60. Memory 44 (e.g., memory unit, memory device, storage device, etc.) may be one or more devices for storing data and/or computer code for completing and/or facilitating the various processes described in the present disclosure. Memory 44 may include volatile memory and/or non-volatile memory. Memory 44 may include database components, object code components, script components, and/or any other type of information structure for supporting the various activities described in the present disclosure.

According to an exemplary embodiment, any distributed and/or local memory device of the past, present, or future may be utilized with the systems and methods of this disclosure. According to an exemplary embodiment, memory 44 is communicably connected to processing circuit 42 (e.g., via a circuit or any other wired, wireless, or network connection) and includes computer code for executing one or more processes described herein. A single memory unit may include a variety of individual memory devices, chips, disks, and/or other storage structures or systems.

Module or subsystem components 46, 50-60 may be computer code (e.g., object code, program code, compiled code, script code, executable code, or any combination thereof) for conducting each module's respective functions. Module components 46, 50-64 may be stored in memory 44, or in one or more local, distributed, and/or remote memory units configured to be in communication with processing circuit 42 or another suitable processing system.

Communication interface 48 includes one or more component for communicably coupling controller 40 to the other components of catheter procedure system 10 via communication links 38. Communication interface 48 may include one or more jacks or other hardware for physically coupling communication links 38 to controller 40, an analog to digital converter, a digital to analog converter, signal processing circuitry, and/or other suitable components. Communication interface 48 may include hardware configured to connect controller 40 with the other components of catheter procedure system 10 via wireless connections. Communication module 46 is configured to support the communication activities of controller 40 (e.g., negotiating connections, communication via standard or proprietary protocols, etc.).

Data storage module 58 is configured to support the storage and retrieval of information by controller 40. In one embodiment, data storage module 58 is a database for storing patient specific data, including image data. In another embodiment, data storage module 58 may be located on hospital network 34. Data storage module 58 and/or communication module 46 may also be configured to import and/or export patient specific data from hospital network 34 for use by controller 40.

Controller 40 also includes a procedure control module 50 configured to support the control of bedside system 12 during a catheter based medical procedure. Procedure control module 50 allows the manipulation of controls 16 by the user to operate bedside system 12. Procedure control module 50 may also cause data appropriate for a particular procedure to be displayed on monitors 26 and 28. Procedure control module 50 may include sets of instructions specific to various types of catheter based procedures that may be performed using bedside system 12. For example, procedure control module 50 may include one set of instructions that will be executed by processing circuit 42 if bedside system 12 is being used to perform a diagnostic catheterization procedure and another set of instructions that will be executed by processing circuit 42 if bedside system 12 is being used to perform an therapeutic catheter procedure. In addition, procedure control module 50 may also be configured to allow a user located at workstation 14 to operate imaging system 32.

Controller 40 also includes simulation module 52. Simulation module 52 is configured to run a simulated catheterization procedure based upon stored vascular image data and also based upon a user's manipulation of controls 16. In one embodiment, simulation module 52 generates a display as a function of the vascular image data and causes the display of a simulated percutaneous intervention device within the vascular image data as a function of the user input allowing the user to perform a simulated catheterization procedure. The vascular image data may be patient specific data (e.g., image data representing a patient's heart) or it may be a simulated vascular image. Generally, simulation module 52 is configured to allow a user to manipulate controls 16 during the simulated procedure, to determine how bedside system 12 would respond to the manipulation of these controls if the procedure were not simulated, and, based upon this, to provide feedback to the user regarding the user's performance during the simulated procedure. In one embodiment, controller 40 is part of a stand alone simulation device not communicably coupled to bedside system 12.

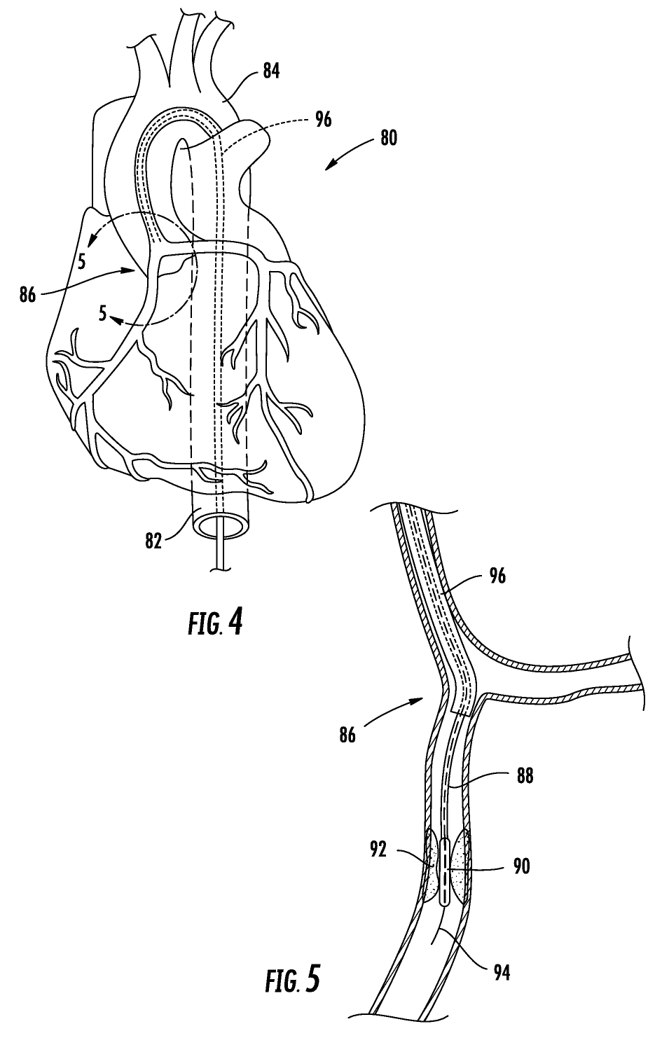

Referring to FIGS. 4-5, a simulated catheter based medical procedure based upon patient specific data and user inputs is shown according to an exemplary embodiment. In this embodiment, simulation module 52 is configured to cause the display of patient specific data, shown as stored image 80 of a patient's heart, onto monitor 26 and/or 28. Image 80 shows the aorta 82, aortic arch 84, and the coronary arteries 86. Simulation module 52 is also configured to cause an image of one or more virtual or simulated percutaneous intervention devices, shown as virtual working catheter 88, virtual guide wire 94, and/or virtual guide catheter 96, to be displayed within image 80. As shown in FIGS. 4-5, the image virtual working catheter 88, virtual guide wire 94, and virtual guide catheter 96 are displayed within the aorta and/or coronary arteries shown in image 80.

Simulation module 52 is configured to change the display of virtual working catheter 88, virtual guide wire 94, and/or virtual guide catheter 96 relative to image 80 in response to the user's manipulation of controls 16. This allows the user to practice the manipulation of controls 16 to position virtual working catheter 88, virtual guide wire 94, and/or virtual guide catheter 96 into the proper position as needed for a particular procedure and for a particular patient. FIG. 5 shows the positioning of virtual working catheter 88, virtual guide wire 94, and virtual guide catheter 96 during a simulated balloon angioplasty therapeutic catheterization procedure. As shown in the exemplary procedure of FIG. 5, the user manipulates controls 16 to position virtual working catheter 88, virtual guide wire 94, and/or virtual guide catheter 96 such that virtual angioplasty balloon 90 is positioned adjacent lesion 92. The user then manipulates controls 16 to inflate virtual angioplasty balloon 90, to deflate virtual angioplasty balloon 90, and to retract virtual working catheter 88, virtual guide wire 94, and/or virtual guide catheter 96. Thus, simulation module 52 allows the user to practice a catheter based procedure based upon the geometry of a particular patient's arterial system while interacting with the same user interface 30 and controls 16 that the user will interact with during a real catheter based procedure. Because simulation module 52 allows the user to practice a procedure “virtually,” surgical time, error rates, etc. may be decreased during the real catheter based procedure.

Simulation module 52 may be configured to provide a simulation of any type of catheter based intervention procedures, such as stent procedures, balloon angioplasty, etc. In addition, simulation module 52 may be configured to provide a simulation of a diagnostic catheterization procedure based upon patient specific data obtained during a previous diagnostic catheterization procedure. In addition, simulation module 52 may be configured to run a simulation based on other forms of patient specific data, such as measurement data. The patient specific data upon which the simulation is based may be stored anywhere, including in controller 40 or on hospital network 34. In addition, patient specific data, such as image 80, may be generated by imaging system 32 associated with catheter procedure system 10. However, patient specific data need not be generated by or stored in a device associated with catheter procedure system 10 as long as catheter procedure system 10 has access to the data at the time of the simulation. In addition, simulation module 52 may be used to perform a simulation for general training purposes (i.e., not practice for a specific procedure to be performed on a particular patient).

In one embodiment, patient specific data is image data of a patient's heart taken during a catheter based diagnostic procedure. In another embodiment, patient specific data is 3D image data of a patient's heart. The 3D image data may be generated by any 3D imaging modality of the past, present, or future, such as an x-ray based computed tomography (CT) imaging device, a magnetic resonance imaging device, a 3D ultrasound device, etc. In another embodiment, patient specific data is image data generated by an IVUS system.

In one embodiment, simulation module 52 determines the response of a virtual component to the user's manipulation of controls 16 and determines the position of the virtual component relative to the three dimensional (“3D”) image data based upon the response of the virtual component. In this embodiment, simulation module 52 may be configured to display a two dimensional (“2D”) image based upon the 3D image data to mimic a 2D real-time image of a patient's heart that may be displayed during the real catheterization procedure. In this embodiment, simulation module 52 displays an image of the virtual component over the 2D image, however, the 2D display of the virtual component within the 2D image of the heart takes into account the position of the virtual component relative to the 3D image data. For example, if the virtual component moves perpendicular to the plane of the 2D image, the image of the virtual component will change in size.

In one embodiment, simulation module 52 is configured to display 2D images during the simulated procedure that represent different angular views of the real-time images that the user may select to be displayed during a real catheterization procedure. In one embodiment, during the simulated procedure, the user may select or rotate through different angular views to determine which angular 2D view is suited for display during a particular portion of the procedure. In another embodiment, simulation module 52 may be configured to display the virtual component positioned within a 3D image of the patient's heart to mimic a 3D real-time image of a patient's heart that may be displayed during the real catheterization procedure.

In another embodiment, simulation module 52 is configured to display a 2D image in one portion of the screen and a 3D image in another portion of the screen. In this embodiment, the 2D image mimics a 2D real-time image of the patient's heart that may be displayed during a real catheter based procedure, and the 3D image will allow the user to operate controls 16 to move the virtual component through the 3D image while observing the movement of the virtual component in the corresponding 2D image.

In one embodiment, simulation module 52 is configured to allow the user to perform the simulated procedure with various virtual percutaneous intervention devices to help the user decide which percutaneous intervention devices will be used during the real catheterization procedure. For example, a user may perform several simulated catheterization procedures with one or more different virtual guide catheters, virtual guide wires, virtual working catheters, etc., and the user may select the percutaneous intervention devices to be used during the real catheterization procedure based upon how well the virtual percutaneous intervention devices performed during the simulated procedure. In addition, for a catheterization procedure that requires the end or tip of the guide wire to bent prior to insertion into the patient, the user may perform the simulation with different bend angles introduced into the end of the virtual guide wire, and the user may introduce a bend angle in to the real guide wire based upon how well the different bend angles performed during the simulated procedure.

In one embodiment, simulation module 52 may be configured to provide feedback to the user regarding the efficacy of a particular treatment in restoring blood flow through the portion of the coronary artery effected by a lesion. In one embodiment, the patient specific data utilized by simulation module 52 includes both image data and blood flow data. In this embodiment, the user may deploy a particular virtual stent at the site of virtual lesion 92, and simulation module 52 will determine the amount of blood flow restored by the deployment of that particular stent. The user then may perform the simulated procedure again with a different virtual stent to determine if a better result may be achieved with the second stent. Based upon this simulation, the user then may perform the real catheterization procedure using a real stent that corresponds to the virtual stent used during one of the simulated procedures. In one embodiment, controller 40 may include an inventory module 55 that is in communication with the hospital's inventory management system or software, to ensure that percutaneous intervention devices selected during the simulated procedure are in inventory or are ordered so that they are available for use during the real catheterization procedure.

In another embodiment, simulation module 52 is configured to simulate any number of problems that may be encountered during a catheter based procedure. Simulation module 52 may be configured to simulate one or more problems that the patient may experience during a catheterization procedure (e.g., heart attack, sudden drop in blood pressure, etc.). In another embodiment, simulation module 52 may be configured to simulate one or more problems that may occur with catheter procedure system 10 during a catheterization procedure (e.g., lock up of controls 16, freeze of monitor 26 or 28, malfunction of bedside system 12, malfunction of imaging system 32, etc.). This may allow the user to train for a variety of problems that may be experienced during the use of catheter procedure system 10.

In one embodiment, simulation module 52 may include a randomness generator (e.g., random number generator). The randomness generator may allow simulation module 52 to introduce various random procedural factors into a simulated procedure to simulate randomness that may be experienced during a real catheter based procedure. For example, randomness generator may alter the response that one of the virtual percutaneous intervention devices has to actuation of controls 16 to simulate the effect that blood flow during systole may have on the real component. In one embodiment, simulation module 52 may be configured to allow the user to turn the randomness generator on and off allowing the user to select whether to perform a simulation with or without random effects.

In one embodiment, simulation module 52 requires a user to perform one or more steps to configure simulation module 52 to run a simulation using a particular set of patient specific data. For example, the user may identify a start point within the image data of the patient's heart. In this embodiment, the start point is the position from which simulation module 52 will begin moving the virtual component in response to the user's manipulation of controls 16. In another embodiment, simulation module 52 automatically configures simulation module 52 to run a simulation using a particular set of patient specific data. For example, simulation module 52 may start every simulation from a standard start point, such as the entry to the aortic arch, or, if the virtual component is a virtual guide wire or virtual working catheter, simulation module 52, may automatically start the simulation from the point where the virtual guide wire or virtual working catheter exits the guide catheter.

Controller 40 also includes record module 60. Record module 60 is configured to record procedural data (i.e., data representing various aspects of a simulated catheter based procedure and/or various aspects of a real catheter based procedure). Record module 60 may be configured to record any aspect of a simulated catheter based procedure or a real catheter based procedure, such as image data, patient data, hemodynamic data, amount of contrast agent used, amount and type of medicine delivered, movement and positioning of the percutaneous intervention devices, actuation of the controls, angular views utilized, etc. This recorded information may be used for any number of purposes, such as training, simulation, or diagnosis. In one embodiment the recorded information may be associated and archived with the medical record of a patient. In this embodiment, the information record by record module 60 may be stored in data storage module 58 and/or stored on hospital network 34.

In one embodiment, record module 60 may record images that are displayed on monitors 26 and 28 during a simulated or real catheter based procedure. The recorded images may be viewed at a later time as series of single images or as a movie. The recorded images may be stored in data storage module 58, on hospital network 34, or in any other suitable location. The review of the recorded images from a catheter based procedure may be used for various training purposes (e.g., display in a class room, review and critique of a procedure by a more experienced physician, etc.).

In one embodiment, record module 60 is configured to allow a user to edit the recorded information. For example, if the information recorded by record module 60 includes images of a simulated or real catheter based procedure, record module 60 may be configured to allow the user to delete images that the user believes are not needed. This may allow the user to conserve data storage space on data storage module 58 and/or hospital network 34. In another embodiment, record module 60 is configured to segment or categorize the recorded information. For example, record module 60 may segment a series of images recorded based upon the component that is being controlled by the user in those images. For example, the portion of a procedure during which the user is advancing the guidewire is labeled, flagged, bookmarked, etc. and the portion of the procedure during which the user is inflating a balloon or stent is labeled, flagged, bookmarked, etc. This may allow the user to conveniently select and view only the portions of the procedure that the user would like to review.

In another embodiment, record module 60 may be configured to generate and store a summary of a recorded procedure in addition to and/or in place of the full recorded procedure. In one embodiment, the summary of the recorded procedure may only include images of important, relevant, difficult, etc., portions of the procedure that are most valuable for later review. The important portions of the procedure may be automatically selected and stored as part of the summary by record module 60, or the important portions may be selected by the user to be stored as part of the summary. In addition, the summary may be compressed and/or of lower image quality than is required for medical purposes in order to facilitate storage and transfer of the summary. In one embodiment, the summary may be a series of animated images showing a recorded procedure. In one embodiment, the summary may be associated with the patient's medical records. In another embodiment, the summary (e.g., on a disk, CD, DVD, etc.) may be given to the patient.

In another embodiment, lab unit 11 or workstation 14 may be equipped with an input device, such as a computer, tablet computer, laptop computer, handheld computer, etc., that allows a worker (e.g., physician, nurse, technician, etc.) working within lab unit 11 to enter information about the procedure. Record module 60 may be configured to store this data along with the other procedural information that record module 60 is configured to record. In one embodiment, record module 60 records the time that data was entered by the worker along with the entered data. This may allow record module 60 to correlate the data entered by the worker with the other procedural information record module 60 records. For example, if the worker enters that a certain amount of medicine is given to a patient at time X, the user is able to review image data recorded before, during, and/or after time X to determine what caused the need for the medicine and/or what the effect of the medicine was.

In one embodiment, record module 60 is configured to record particular aspects of a simulated catheter based procedure in order to adjust the operation of catheter procedure system 10 during a real catheter based procedure. In another embodiment, record module 60 is configured to record particular aspects of a catheter based diagnostic procedure in order to adjust the operation of catheter procedure system 10 during a catheter based therapeutic procedure. In one embodiment, procedure control module 50 may utilize certain data recorded during a simulated procedure on a particular patient to adjust how bedside system 12 responds to manipulation of controls 16 during the real procedure on that same patient. For example, if one or more of controls 23, 25, and/or 29, were actuated to advance the associated percutaneous intervention devices for longer than typical amounts of time during the simulated procedure (indicating that the anatomy or geometry of a patient's arterial system is larger than normal), procedure control module 50 may increase the sensitivity of controls 23, 25, and/or 29 during the real catheter based procedure. Alternatively, if controls 23, 25, and/or 29 were actuated for shorter than typical amounts of time during the simulated procedure (indicating that the anatomy or geometry of a patient's arterial system is smaller than normal), procedure control module 50 may decrease the sensitivity of controls 23, 25, and/or 29 during the real catheter based procedure.

In another embodiment, procedure control module 50 may also utilize certain data recorded during a simulated procedure to automate operation of bedside system 12 during a real catheter based procedure. For example, record module 60 may record the distance the virtual catheter was advanced to reach the aortic arch during the simulated procedure, and procedure control module 50 may utilize this data to automatically advance a real catheter to the aortic arch during the real procedure. Procedure control module 50 may be configured to combine automatic operation of bedside system 12 with user input at controls 16 during a real procedure allowing the user to determine whether a particular portion of the procedure may be preformed automatically based on recorded data from a simulated procedure or manually via operation of controls 16.

In other embodiments, controller 40 includes an assist module 53 configured to provide information to the user during a real and/or simulated catheterization procedure to assist the user with the performance of the procedure. The information provided by assist module 53 may be determined based upon the stored patient specific data and/or based upon information recorded during a simulated or real catheter based procedure. In one embodiment, assist module 53 is configured to display images of the recorded simulation procedure during the real catheterization procedure. In one embodiment, the display may be a split screen having the display of images of the recorded simulation procedure on one side of the monitor, and the real-time images of the real catheterization procedure on the other side of the monitor. The display of the images of the simulated procedure may be correlated with the current position of the component that is currently being operated in the real procedure. For example, if the user is advancing the real guide wire into the coronary artery, the display of images from the simulated procedure may show the advance of the virtual guide wire into the coronary artery. This will allow the user to operate controls 16 to move the real guide wire to mimic the movement of the virtual guide wire practiced during the simulated procedure.

In another embodiment, assist module 53 may be configured to display images of the simulated procedure within the real-time images taken during the real catheterization procedure. In this embodiment, assist module 53 may be configured to automatically detect the image of one or more of the real percutaneous intervention devices (e.g., the guide wire, guide catheter, working catheter, balloon, etc.) in the real-time images and to align the image of the corresponding virtual component with the image of the real component. In this embodiment, the user may operate controls 16 during the real catheterization procedure such that the movements of the real component match the movements of the virtual component recorded during the simulation procedure. For example, in one embodiment, assist module 53 is configured to display an image of the expanded simulated stent or balloon at the location of the lesion on top of the real-time images. The user then may expand the real balloon and/or stent so that the image of the real balloon and/or stent is aligned with the image of the expanded simulated balloon and/or stent.

In another embodiment, assist module 53 may be configured to calculate a suggested path for a component based upon the movement of that component during the simulated procedure. In this embodiment, assist module 53 may display the suggested path on top of the real-time images taken during the real catheterization procedure. In one embodiment, if the user deviates away from the suggested path, assist module 53 may be configured to display a suggested correction path that the user may follow to realign the component with the suggested path. In one embodiment, procedure control module 50 may be configured to automatically control bedside system 12 to follow the suggested path. In this embodiment, assist module 53 and/or procedure control module 50 may allow the user to switch from automatic control of the component to manual control of the component via operation of controls 16.

In another embodiment, assist module 53 may determine the manner in which controls 16 may be operated to achieve a particular position within the patient's vascular system. Assist module 53 may be configured to display instructions to the user indicating how the user should manipulate controls 16 to reach the particular position within the patient's vascular system. In one embodiment, touch screen 18 of controls 16 may include icons 162, 164, and 166 displayed above controls 29, 23, and 25, respectively. Assist module 53 may be configured to alter the display of icons 162, 164, and/or 166 in order to indicate that assist module 53 has determined that the user should manipulate the respective controls 29, 23, and 25. For example, the display of icon 162 may change to indicate that assist module 53 has determined that the user should manipulate guide catheter control 29 in a certain way (e.g., for a certain length of time). In other embodiments, other icons may be displayed to indicate the controls that assist module 53 has determined that the user should manipulate at various points of the procedure.

In another embodiment, one or more of the real percutaneous intervention devices may include a sensor that communicates the position of the component within the patient's vascular system to controller 40. The position information from the sensor allows assist module 53 to display an image of the real component within the stored 3D image of the patient's heart to represent the position of the component in the patient's heart during a real catheterization procedure. In this embodiment, a 3D representation of the real catheterization procedure may be displayed to the user, even if imaging system 32 is not capable of capturing a real-time 3D image of the patient's heart during the procedure. In one embodiment, bedside system 12 includes one or more encoders that measure the distance that a particular component has been advanced into the patient's vascular system during a real catheterization procedure. Assist module 53 may use the data from the encoders to aid in the location of the component within the 3D image.

In another embodiment, assist module 53 may be configured to aid the user of workstation 14 to select the angular positioning of imaging system 32 relative to patient 21 so that the patient's heart is imaged from the desired angle and that proper angular views of the patient's heart are displayed during the real catheterization procedure. In this embodiment, at least one angular view of the heart of the patient captured by the imaging system during a real catheterization procedure generally approximates (i.e., similar size, magnification, angular view, etc. taking into account noise, patient motion, variability in position, etc.) at least one two dimensional image displayed by the simulation subsystem. In this embodiment, assist module 53 may utilize the angular views that the user selected during the simulated procedure and/or may determine the optimal angular views for particular portions of the procedure based upon an analysis of the stored image data of the patient's heart.

In one embodiment, assist module 53 may be configured to automatically rotate imaging system 32 on the C-arm to obtain images at the proper angular position for a particular portion of the procedure. In another embodiment, assist module 53 may inform or remind the user of workstation 14 during the real catheterization procedure of the views the user used or selected for various portions of the procedure during the simulated procedure, and the user rotates (e.g., manually, through control of the C-arm by manipulation of controls 16, etc.) imaging system 32 to obtain the desired angular views of the patient's heart for the particular portions of the procedure. The reminder generated by assist module 53 may be in any form, such as graphical, textual, verbal, etc.

In another embodiment, assist module 53 may be configured to alert the user to particular problems that were encountered during a simulated procedure. During the simulated procedure, record module 60 may record or flag certain aspects of the simulated procedure when a problem or difficulty is encountered. Record module 60 may identify a problem based upon certain events that indicate that the user is experiencing difficulty during a particular portion of the procedure. For example, if one or more of the virtual percutaneous intervention devices contact a wall of an artery at a particular point, if forward progress of the virtual component stops at a certain point despite actuation of controls 16, if the user reverses and advances the virtual component one or more times at a particular position during the procedure, etc., record module 60 may flag the point or area associated with the problem. In one embodiment, assist module 53 may highlight the problem area on the display of the patient's heart captured during the real catheterization procedure and may suggest how to avoid the problem.

In another embodiment, assist module 53 may be configured to suggest specific (e.g., specific size, shape, function, brand, material, etc.) percutaneous intervention devices for the user to use during a real procedure based upon the patient specific data and/or the performance of the simulated procedure. In one embodiment, assist module 53 may suggest that a particular size catheter, guide wire, angioplasty balloon, stent, etc. should be used based upon an evaluation of the size or geometry of the relevant portions of the patient's vascular system. For example, assist module 53 may automatically calculate the width of the coronary artery at the site of a lesion and suggest a specific stent that would be appropriate for that particular lesion. In another embodiment, assist module 53 may suggest that a particular size catheter, guide wire, angioplasty balloon, stent, etc. should be used based upon the performance of the simulated procedure. For example, if the user had difficulty navigating a narrow portion of or a sharp turn in the coronary artery, assist module 53 may suggest a catheter having a width suited for navigation through that portion of the coronary artery.

In one embodiment, assist module 53 may be configured to learn a particular physician's preferences for specific percutaneous intervention devices based upon the percutaneous intervention devices that the physician selects to use during real and/or simulated procedures. In this embodiment, assist module 53 may suggest specific percutaneous intervention devices based at least in part on the physician's preferences. In another embodiment, assist module 53 may be configured to remind the physician of specific percutaneous intervention devices that were selected by the physician based upon the simulated procedures. In another embodiment, assist module 53 may be configured to remind the physician of the specific bend angle to be introduced into the guide wire that was determined during the simulated procedures. In one embodiment, inventory module 55 is in communication with the hospital's inventory management system or software to ensure that percutaneous intervention devices suggested by assist module 53 are in inventory or are ordered so that they are available for use during the real catheterization procedure.

In another embodiment, assist module 53 is configured to assist in and/or optimize image acquisition during the real catheterization procedure. For example, assist module 53 may be configured to determine the amount of contrast agent and/or radiation that will need to be delivered to the patient during the real catheterization procedure. In another embodiment, assist module 53 may be configured to time the delivery of contrast agent and/or radiation to minimize the contrast agent dose and/or radiation dose. In one embodiment, assist module 53 may be configured to suggest percutaneous intervention devices for use during the real catheterization procedure that will allow for the minimization of contrast agent and/or radiation delivered to the patient.

In another embodiment, assist module 53 may be configured to delay and/or alter movement of a component in response to operation of controls 16 in order to optimize the movement and/or control of the component. In one embodiment, assist module 53 may delay the movement of a component in response to operation of controls 16 based upon the stage of the patient's heart beat, breathing, etc. For example, if the user operates controls 16 to advance a component during a portion of the heart beat cycle when the response of that component is likely to be effected, assist module 53 may delay movement of the component until a portion of the heart beat cycle when the response of the component is not likely to be effected. In one embodiment, if the user operates controls 16 to move a component during the systolic phase of the heart, assist module 53 may be configured to delay the movement of a component in response to the operation of controls 16 until the heart enters diastole.

The information provided to the user by assist module 53 may be adjusted to account for variables present in the patient specific information or in the recorded procedural information. In one embodiment, assist module 53 may be configured to account for various errors contained in the image date that assist module 53 utilizes to provide any of the assistance discussed above. For example, assist module 53 may compensate for noise contained in the image data, the resolution of the imaging system, movement caused by the beating of the patient's heart, movement caused by the patient's breathing, etc.

Referring again to FIG. 3, controller 40 may be operated in at least a first mode and a second mode. In the first mode or procedure mode, controller 40 operates as directed by procedure control module 50 allowing the user to control and/or operate bedside system 12 via manipulation of controls 16. In the second mode or simulation mode, controller 40 operates as directed by simulation module 52 allowing the user to control virtual working catheter 88, virtual guide wire 94, and/or virtual guide catheter 96 via manipulation of controls 16. Controller 40 also includes a mode selection module 54 that allows the user to switch controller 40 between the first and second modes.

In another embodiment, selection module 54 allows the user to switch between the procedure mode and the simulation mode while the user is performing a real catheterization procedure. When the user switches to simulation mode during a real procedure, controls 16 will cease to control bedside system 12 and simulation module 52 will run a simulated catheterization procedure as discussed above. In one embodiment, simulation module 52 will display an image that corresponds to the current position of the real percutaneous intervention devices within the vascular system of the patient. The user may then practice the next series of movements by operating a virtual component via manipulation of controls 16. Following the simulation, the user then may switch back to procedure mode allowing the user to operate the real component via manipulation of controls 16. This embodiment allows the user to practice a particular portion of the procedure immediately before performing that portion of the procedure on the patient.

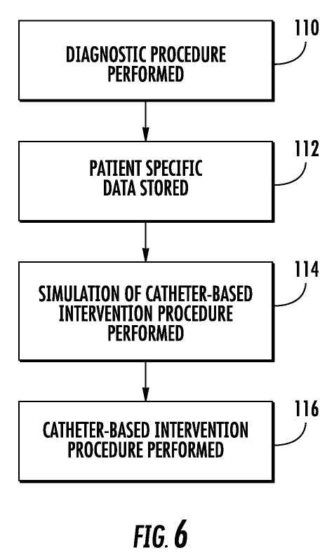

FIG. 6 shows a flow-chart of a catheter based procedure according to an exemplary embodiment. At step 110, a diagnostic procedure is preformed. In one embodiment, a diagnostic catheter based procedure may be preformed at step 110.

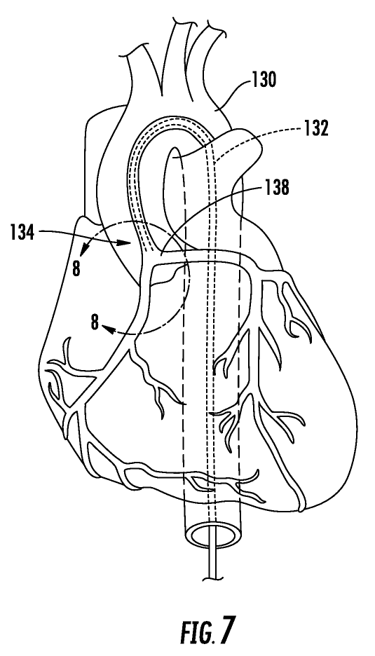

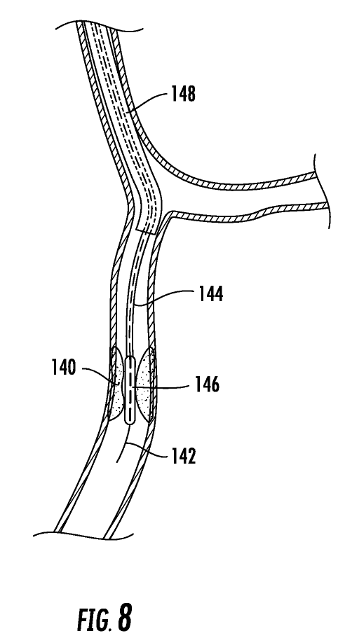

Referring to FIG. 7, a human heart is shown during a diagnostic catheterization procedure. During an exemplary diagnostic catheterization procedure, an incision is made, usually in the groin. A 0.038 guide wire (not shown) is inserted through the incision and into the femoral artery. Bedside system 12 is operated to feed the 0.038 guide wire through the patient's arterial system and over the top of aortic arch 130. Bedside system 12 is operated to advance a diagnostic catheter 132 over the 0.038 guide wire until diagnostic catheter 132 is positioned near the intersection between the aorta and coronary arteries shown at 134. The 0.038 guide wire is removed allowing diagnostic catheter 132 to return to a preformed shape enabling the diagnostic catheter 132 to access either the left ostium 136 or the right ostium 138 of the aorta. In one embodiment, three diagnostic catheters are used during step 110. Each of the three catheters is shaped differently to allow access to the left ostium, the right ostium, and the chambers of the heart, respectively. A contrast media is injected through the diagnostic catheter 132. Imaging system 32 is then operated to image the patient's heart and coronary arteries to identify the existence and location of any lesion, such as atherosclerosis 140 (shown in FIG. 8). After the image(s) are captured, the 0.038 guide wire and diagnostic catheter 132 are removed and the incision is closed. The patient then may be kept at the medical facility that conducted the diagnostic procedure, the patient may be sent home if the patient's condition allows, or the patient may be sent to another facility to undergo a catheter based therapeutic procedure.

In another embodiment, step 110 may include non-catheter based diagnosis procedures. For example, diagnostic image data may generated during a non-catheter based x-ray, CT, MRI, ultrasound, etc. In another embodiment, other patient specific data such as anatomical measurements, hemodynamic data, catheter or guide wire position or movement data, etc. may be captured during the diagnostic procedure of step 110. In another embodiment, step 110 may comprise an earlier catheter based therapeutic procedure during which patient specific data is generated.

At step 112 patient specific data generated during step 110 , such as the image data generated, is retrievably stored for later use. The patient specific data may be stored in data storage module 58 of controller 40. Alternatively, the patient specific data may be stored on a hospital data management system or on hospital network 34. This data may be stored as part of database to associate the data with a particular patient and/or a particular procedure. This data may be stored in any manner that allows the data to be used during a simulation of a catheter based procedure.

At step 114, a simulation of a catheter based therapeutic procedure (e.g., angioplasty, peripheral vascular intervention, etc.) is preformed based upon the patient specific data generated in step 110 and stored in step 112. As discussed above, the simulation may be preformed based upon the image data of the patient's heart obtained during step 110. In an exemplary embodiment, step 114 is preformed following the diagnostic procedure and after the patient has been disconnected from catheter procedure system 10. In another embodiment, step 114 may be performed during step 116.

At step 116, a catheter-based therapeutic procedure is performed. Referring to FIG. 8, a coronary artery having a lesion 140 is shown during a catheter based therapeutic procedure. During the exemplary balloon angioplasty intervention procedure of FIG. 8, an incision is made, usually in the groin. A guide catheter 148 is inserted through the incision into the femoral artery. Bedside system 12 is operated to feed guide catheter 148 through the patient's arterial system until guide catheter 148 is positioned near either the left or right ostium. Bedside system 12 is then operated to feed guide wire 142 through guide catheter 148 until guide wire 142 extends across lesion 140. Next, bedside system 12 is operated to advance working catheter 144 over guide wire 142 to position balloon 146 across lesion 140. Once working catheter 144 and balloon 146 is in place, balloon 146 is inflated to compress lesion 140 and to stretch the artery open thereby increasing blood flow to the heart. Balloon 146 is then deflated, guide wire 142 and working catheter 144 are removed, and the incision is closed.

While the catheter based therapeutic procedure discussed above relates to a balloon angioplasty, it should be understood that the catheter used during step 116 may be any type of catheter useful during the performance of any percutaneous procedure. For example, the catheter may include a stent that is expanded and left at the site of the lesion. Alternatively, the catheter may include structures adapted to cut or grind away the plaque forming the lesion.

The exemplary embodiments illustrated in the figures and described herein are offered by way of example only. Accordingly, the present application is not limited to a particular embodiment, but extends to various modifications that nevertheless fall within the scope of the appended claims and also extends to any combination of the features or elements described herein or set forth in the claims.

The construction and arrangement of the systems and methods as shown in the various exemplary embodiments are illustrative only. Although only a few embodiments have been described in detail in this disclosure, many modifications are possible (e.g., variations in sizes, dimensions, structures, shapes and proportions of the various elements, values of parameters, mounting arrangements, use of materials, colors, orientations, etc.). For example, the position of elements may be reversed or otherwise varied and the nature or number of discrete elements or positions may be altered or varied. All such modifications are intended to be included within the scope of the present disclosure.

The order or sequence of any process or method steps may be varied or re-sequenced according to alternative embodiments. Also two or more steps may be performed concurrently or with partial concurrence. Such variation will depend on the software and hardware systems chosen and on designer choice. All such variations are within the scope of the disclosure. Likewise, software implementations could be accomplished with standard programming techniques with rule based logic and other logic to accomplish the various connection steps, processing steps, comparison steps and decision steps. Other substitutions, modifications, changes, and omissions may be made in the design, operating conditions and arrangement of the exemplary embodiments without departing from the scope of the present disclosure.

What is claimed is:

1. A workstation configured for both operating and simulating a robotic catheter system and interventional procedure, comprising:
a user interface configured to receive a user input;
a control system operatively coupled to the user interface, the control system configured to remotely and independently control at least two percutaneous intervention devices during a real catheterization procedure;
a data storage subsystem storing vascular image data; and
a simulation subsystem configured to simulate a catheterization procedure;
wherein the control system controls the at least two percutaneous intervention devices based upon the user input received by the user interface to allow the user to perform the real catheterization procedure; and
further wherein the simulation subsystem generates a display as a function of the vascular image data and causes the display of a simulated percutaneous intervention device within the display of vascular image data as a function of the user input allowing the user to perform the simulated catheterization procedure.

2. The workstation of claim 1 wherein the vascular image data is patient specific image data.

3. The workstation of claim 1 wherein the vascular image data comprises an image of a heart.

4. The workstation of claim 1 wherein the vascular image data is three dimensional image data.

5. The workstation of claim 4 wherein the simulation subsystem displays a two dimensional image generated from the three dimensional image data and causes the display of the simulated percutaneous intervention device within the display of the two dimensional image, and further wherein the display of the simulated percutaneous intervention device within the two dimensional image changes as a function of the user input.

6. The workstation of claim 4 wherein the simulation subsystem displays both a three dimensional image as a function of the three dimensional image data and a two dimensional image generated from the three dimensional image data.

7. The workstation of claim 1 further comprising:
a first mode, wherein the control system is active in the first mode;
a second mode, wherein the simulation subsystem is active in the second mode; and
a mode selection subsystem allowing the user to select between the first mode and the second mode.

8. The workstation of claim 7 wherein the mode selection subsystem allows the user to switch from the first mode to the second mode during a real catheterization procedure.

9. The workstation of claim 1 further comprising an assist subsystem wherein the assist subsystem provides the user with information during a real catheterization procedure to assist the user in the performance of the catheterization procedure.

10. The workstation of claim 9 wherein the information provided by the assist subsystem is a function of the vascular image data.

11. The workstation of claim 10 wherein the information provided comprises a list of percutaneous intervention devices.

12. The workstation of claim 9 wherein the data storage subsystem stores recorded procedural data, and further wherein the information provided by the assist subsystem is a function of the recorded procedural data, and further wherein the information provided by the assist subsystem includes a suggested path as a function of the recorded procedural data.

13. The workstation of claim 1 wherein the simulation subsystem allows the user to perform the simulated procedure with a plurality of simulated percutaneous intervention devices.

14. The workstation of claim 13 wherein the control system is in communication with a hospital inventory system, the hospital inventory system including a list of percutaneous intervention devices available for use during the real catheterization procedure, wherein the control system displays information identifying percutaneous intervention devices that are included in the list of percutaneous intervention devices or that were used during a simulated catheterization procedure.

15. The workstation of claim 13 wherein the control system is in communication with the hospital inventory system, the hospital inventory system including a list of percutaneous intervention devices available for use during the real catheterization procedure, wherein the simulation subsystem displays information identifying percutaneous intervention devices that are included in the list of percutaneous intervention devices and allows the user to perform a simulated procedure using a percutaneous intervention device that is included in the list of percutaneous devices.

16. A workstation configured for operating and simulating a robotic catheter system and configured for simulating an imaging system movable between a plurality of angular positions relative to a patient to capture a plurality of angular views of a portion of the patient, the workstation comprising:
a user interface configured to receive a user input;
a control system operatively coupled to the user interface configured to remotely and independently control at least two percutaneous intervention devices;
a data storage subsystem to store three dimensional patient specific vascular image data; and
a simulation subsystem configured to simulate a catheterization procedure, wherein the simulation subsystem displays a plurality of two dimensional images generated from the three dimensional vascular image data representative of different angular views of a portion of the patient that may be displayed during a real catheterization procedure;
wherein the control system controls the at least two percutaneous intervention devices based upon the user input received by the user interface to allow the user to perform a real catheterization procedure.

17. The workstation of claim 16 further comprising a procedure record subsystem that records two dimensional images displayed by the simulation subsystem during the simulated catheterization procedure.

18. The workstation of claim 17 wherein the control system displays two dimensional images during a real catheterization procedure that were recorded by the procedure record subsystem during the simulated procedure.

19. The workstation of claim 16 wherein the control system is operatively coupled to an imaging system, the control system controlling the movement of the imaging system between the plurality of angular positions relative to a patient to capture a plurality of angular views of a portion of the patient during a real catheterization procedure;
wherein at least one angular view of the portion of the patient captured by the imaging system during a real catheterization procedure generally approximates at least one two dimensional image displayed by the simulation subsystem.

20. The workstation of claim 19 wherein the control system controls the movement of the imaging system between the plurality of angular positions based upon a second user input received by the user interface.

21. The workstation of claim 19 wherein the control system automatically controls the movement of the imaging system between the plurality of angular positions based upon at least one two dimensional image displayed by the simulation subsystem.

22. A workstation configured for operating and simulating a robotic catheter system and interventional procedure, comprising:
a user interface configured to receive a user input;
a control system operatively coupled to the user interface configured to remotely control at least two percutaneous intervention devices during a real catheterization procedure;
a data storage subsystem to store vascular image data;
a simulation subsystem configured to simulate a catheterization procedure;
wherein the control system controls the at least two percutaneous intervention devices based upon the user input received by the user interface to allow the user to perform the real catheterization procedure;
wherein the simulation subsystem generates a display as a function of the vascular image data and causes the display of a simulated intervention device as a function of the user input; and
further wherein the display generated by the simulation subsystem may be displayed during the real catheterization procedure.

23. The workstation of claim 22 further comprising:
a first mode, wherein the control system is active in the first mode;
a second mode, wherein the simulation subsystem is active in the second mode; and
a mode selection subsystem configured to allow the user to select between the first mode and the second mode;
wherein the mode selection subsystem allows the user to switch from the first mode to the second mode and from the second mode to the first mode during a real catheterization procedure.

24. The workstation of claim 22 further comprising a record subsystem to record procedural information, wherein the record subsystem flags portions of the recorded procedural information associated with a problem encountered during a procedure, and further wherein the control system causes a display of information as a function of the flagged portion of the recorded procedure.
